(19) United States
(12) Patent Application Publication
(10) Pub. No.: US 2020/0111563 A1
(43) Pub. Date: Apr. 9, 2020

(54) Method to help ensure correct medical procedure is performed and billed

(71) Applicant: Akhil Aggarwal, Dayton, OH (US)

(72) Inventor: Akhil Aggarwal, Dayton, OH (US)

(21) Appl. No.: 16/362,684

(22) Filed: Mar. 24, 2019

Related U.S. Application Data
(60) Provisional application No. 62/742,466, filed on Oct. 8, 2018.

(51) Int. Cl. G16H 40/20; G06Q 10/10; G16H 10/60; G06Q 30/04

(52) U.S. Cl. CPC G16H 40/20; G06Q 10/10; G06Q 40/08; G06Q 30/04; G16H 10/60

(57) ABSTRACT

A computerized method for identification and notification of discrepancies in a medical setting from the time a medical provider orders a medical procedure to the time a medical claim is submitted. The computerized method focuses on the following discrepancies:
1. Procedure scheduled is different from the procedure ordered by the medical provider
2. Procedure obtained Authorization for from the medical insurance company is different from the procedure ordered by the medical provider.
3. The Procedure Authorization is obtained under a diagnosis code different from the one under which the procedure was ordered by the medical provider.
4. The Procedure Authorization is obtained for a CPT (Current procedural terminology) code different from the one under which the procedure was ordered by the medical provider.
5. The Date of service (DOS) does not fall within the Procedure Authorization date span.
6. The patient medical insurance is different from the medical insurance from which the authorization was obtained,
7. The diagnosis code documented on the Operative note does not include the diagnosis code under which the procedure was ordered.
8. The CPT code on the medical claim is different from the CPT code for which the authorization was obtained from the medical insurance.
9. The medical insurance name on the medical claim is different from the medical insurance from which the authorization was obtained.
10. The Procedure authorization number on the medical claims is different from the authorization number obtained from the medical insurance.
11. The Date of service on the claim is different (does not fall within) the Procedure Authorization date span.

The computer program will make a recommendation to the user as part of notification on identification of an error. The computer program user can Override the recommendation. The program user will need to add Notes if she/he chooses to override the error generated. All the data related to Override will be logged along with details related to user, notes etc so that medical facility management can review user activity.

### BACKGROUND OF THE INVENTION

### Field of the Invention

The invention relates to medical billing for procedures, surgeries, imaging, lab testing as well as durable medical equipments.

### Related Art

When a patient visits a doctor's office the whole visit is documented in an Encounter. An Encounter mainly comprises of Chief complaint, History of present illness, Review of systems, Physical exam and an Assessment and Plan section. It is in the Assessment and Plan section where the doctor usually documents the treatment plan, diagnosis for the patient and associates orders (e.g. a procedure order named Epidural Caudal to treat Low back pain (ICD 10 code M545). This medical order sets in motion a process which mainly comprises of the following:
a) A receptionist schedules an appointment for the patient to get the ordered procedure done
b) Authorization specialist(s) obtains Prior Authorization/pre certification for the procedure from patient's insurance (usually includes an authorization number (e.g. 098A89), diagnosis code (ICD 10 e.g. M545), cpt code (e.g. 62323) a date span (e.g. Aug. 1, 2018 to Sep. 30, 2018)
c) A receptionist Checks in the patient on the day of scheduled appointment
d) Procedure is performed and documented by Medical provider(s)
e) Procedure is billed out to the patient's insurance by a coder/biller

As clear from the brief description of the process that takes place from the time a procedure is ordered to the time it is billed out to a healthcare insurance, a lot of people are involved in the process (medical provider, receptionist, authorization specialist, coder/biller) and a lot of data is being moving from one stage to next. The process can be divided into the following 4 stages:
1. Order
2. Authorization
3. Operative Note
4. Claim

### BRIEF SUMMARY OF THE INVENTION

The invention comprises of a system and method that ensures data integrity from the Order (e.g. procedure order) to Medical Claim creation resulting in prevention of unnecessary procedures (even imaging or DME) and losses . . . this in turn leads to better patient care and revenue for the medical practice/hospital.

### BRIEF DESCRIPTION OF THE DRAWINGS

FIG. 1 (On Page 10) Depicts retrieval of data from Electronic Health record system to the invented computer program through means of an Application Program Interface (API) call.

Datasheet 1 (On Page 11) depicts the possible error scenarios related to Order (Stage 1) and Authorization (Stage 2). Each row depicts a separate possible error scenario.

Datasheet 2 (On Page 12) depicts the possible error scenarios related to Operative Note documentation (Stage 3) and Claim (Stage 4). Each row depicts a separate possible error scenario.

### DETAILED DESCRIPTION OF THE INVENTION

The invention is based on the philosophy that one way to attain better healthcare results for lower cost is by ensuring the non clinical part is also handled in a clinical fashion. There are several points in this whole process where mistakes can be made which can adversely affect patient's health and drive up the cost. The key components that this invention focuses on are as following:
1. Diagnosis code (ICD 10 code)
2. CPT code
3. Authorization/referral number
4. Authorization date span
5. Healthcare insurance name

The invention is intended to be used with a Electronic Health record (EHR/EMR) system. The intent of the invention is not to automate the process but to help people involved in the process do their tasks better. There are several scenarios where mistakes can be prevented by the invention.

The first set of scenarios focuses on the data from the stages 1 and 2 i.e. Order and Authorization respectively. As part of the invention, a lookup table called Order-CPT lookup table (sample on Page 13) is created that associates each type of Order during a doctor office visit to a CPT code.

Before the scheduled start time (e.g. 9 am) on the day of the procedure appointment (e.g. Sep. 18, 2018) the computer program is run to check on the following data items and notifies the user of discrepancies:

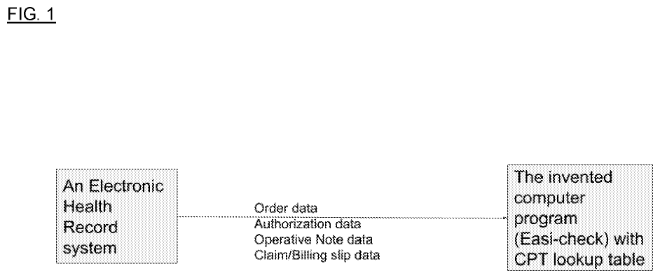

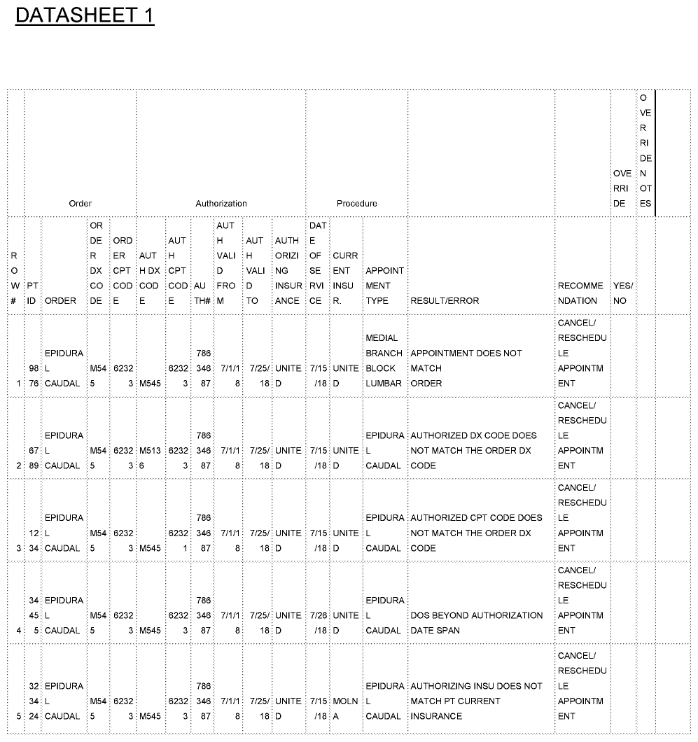

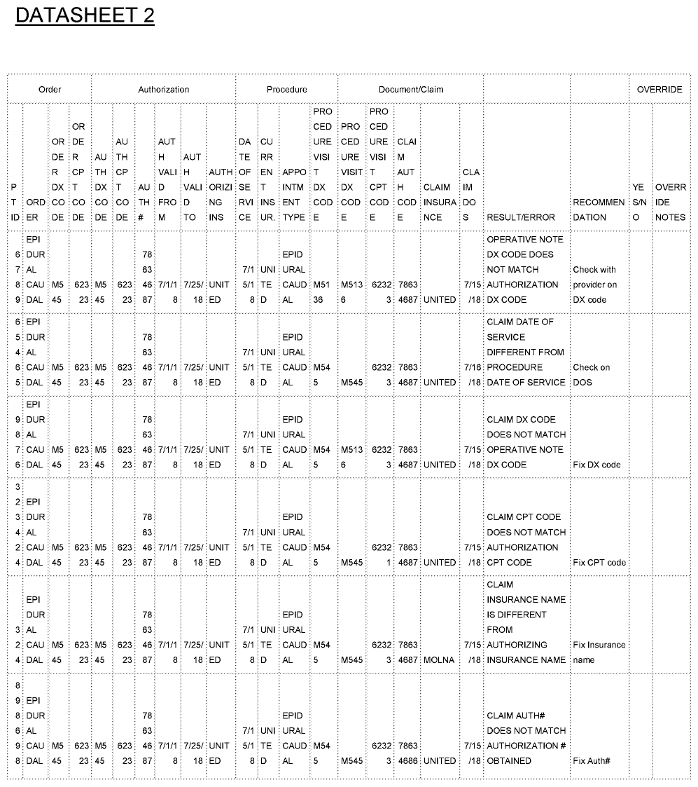

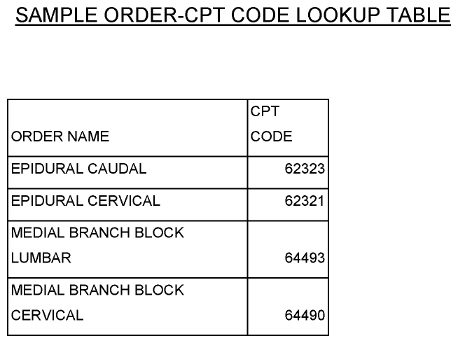

FIG. 1

The computer program will make Application program Interface (API) call(s) to the EHR to get the following data:
1. The procedure orders and their associated Diagnosis codes from the last 3 (or a pre decided number of visits based on the medical specialty) doctor office visits of the patient.
2. The appointment data for a date of service (Sep. 18, 2018) at the medical facility where the procedure will be performed.
3. The Authorization/ Pre certification data which includes at least the Authorization number (e.g. 0467987), Authorization date span (Sep. 1, 2018 to Sep. 18, 2018), Authorization CPT code (e.g. 62323) and Authorization Diagnosis code (e.g. M54.5).

With the help of the Order-CPT lookup table the computer program will associate a CPT code to the Office visit data that has been obtained through the API call to an EHR.

Datasheet 1 Row 1

Illustrates the scenario where Appointment type (e.g. Lumbar Radio Frequency) is different from the Order (Epidural Caudal). This is a common mistake as patients schedule their procedure appointments while checking out at the doctor's office and a receptionist can select a different appointment type than what has been ordered under the Encounter note by the Physician. The intent is to ensure that the patient receives care as per the treatment plan created by the provider and prevent wastage of resources.

The computer program will search for the Appointment type obtained for a patients on the schedule within all the Orders from the last 3 office visits. If no match is found then an error will be generated (e.g. No matching Order found). The computer program user can then cancel/reschedule the appointment or override the error.

Datasheet 1 Row 2

Illustrates the scenario where the Diagnosis code (e.g. M47.896) under which the the Authorization number (e.g. 0467987) has been obtained is different from the one (e.g. M54.5) under which Order (e.g. Epidural Caudal) was placed by a medical provider. An Authorization specialist manually enters the Diagnosis code on the paper/electronic form for obtaining authorization from the patient's healthcare insurance company and hence manual errors are possible. With the introduction of ICD 10 this has become more common since the diagnosis codes are now more specific and discrepancy of even one digit (e.g. M7061 relates to right side hip and M7062 relates to left side hip) can affect patient care.

The computer program will compare the Diagnosis code from office visit data with the Diagnosis code from the authorization data and will generate an error if they are different.

Datasheet 1 Row 3

Illustrates the scenario where CPT code associated with the Authorization data is different from the CPT code associated with the Order from the office visit data.

As mentioned in FIG. 1, this will be possible with the help of the Order-CPT lookup table where the computer program will associate a CPT code to the Office visit data that has been obtained through the API call to an EHR.

Datasheet 1 Row 4

Illustrates the scenario where date of service (e.g. Sep. 18, 2018) does not fall within Authorization date span (Sep. 1, 2018 to Sep. 15, 2018). This usually happens because patients need to reschedule their appointment and the staff member overlooks the Authorization date span while rescheduling.

The computer program user can run the program for all the scheduled patients for a date of service in one instance to see all the discrepancies for the day. There is one more scenario possible for discrepancy and that can be identified by running the computer program on check in of every patients.

Datasheet 1 Row 5

Illustrates the scenario where Healthcare insurance company that provided the Authorization (e.g. United Healthcare) is different from the one that patient has on the date of service (e.g. Anthem). A patient can change their Healthcare insurance frequently, even every few months. Hence a patient might have healthcare coverage from a different insurance company on the scheduled procedure date than what the patient had during doctor office visit. The procedure authorization is also obtained from the same insurance company as the one patient had for office visit until and unless the Authorization specialist finds out during communication with insurance company that patient is no longer eligible for the same insurance.

The second set of scenarios focuses on the data from the stages 3 and 4 i.e. Operative note and Claim respectively. The computer program will make another Application program Interface (API) call(s) to the EHR to get the following data:
1. The procedure documentation data especially the Diagnosis codes from the Operative note
2. The claim/billing slip data for a date of service (Sep. 18, 2018) at the medical facility where the procedure will be performed.

Before the submitting the medical claims for a date of service to the insurance companies, the computer program is run to check on the following data items and notifies the user (medical coder/biller) of the discrepancies:

Datasheet 2 Row 1

Illustrates the scenario where Diagnosis code (M47.896) documented in the Operative note is different from the Authorization Diagnosis code (M54.5). This is entered manually on the Operative note and hence there is always a possibility of error.

Datasheet 2 Row 2

Illustrates the scenario where Date of service (DOS) entered on the claim is different from the Procedure data of service. This can lead serious legal implications for the medical practice.

Datasheet 2 Row (3 to 6)

Illustrates the scenario where data that is manually entered on the claim is different from the Order, the Authorization or the Operative note. The computer program will generate error if it finds the following claim data items are different from Authorization data:
1. Diagnosis code (ICD-10) (e.g. M546) entered on the claim (Row 2)
2. CPT code entered on the claim (e.g. 62321) (Row 3)
3. Medical Insurance name entered on the claim (e.g. Cigna) (Row 4)
4. Authorization code entered on the claim (e.g. 101) (Row 5)

What is claimed is:

1. A computer program that can detect and notify a medical facility staff of the discrepancy between the Medical procedure ordered for a patient and the medical procedure that the patient is scheduled for. The computer program will achieve this by running Application program Interface calls to the Electronic Health record system and obtaining the Procedure order data and Appointment Type data. The computer program will compare the two sets of data for all the patients scheduled for a day at the medical facility.
The computer program of claim 1 can further detect and notify any discrepancy between the Authorization data and Appointment data as following:
a) Authorization date span has expired (e.g. Patient comes to the medical facility on October 4th when the Authorization date span was September 1st to September 30th).
b) Patient medical insurance is different from the medical insurance that Authorization was obtained from.
c) The diagnosis code (ICD 10 code) under which the procedure was ordered is different from the diagnosis code under which the authorization was obtained.
d) The CPT code under which the procedure was ordered is different from the CPT code under which the authorization was obtained.
The computer program of claim 1 can additionally detect and notify if the diagnosis code (ICD 10 code) documented on the Operative note is different from the diagnosis code under which the procedure was originally ordered by the provider.
The computer program of claim 1 can further detect and notify of the following discrepancy between the Authorization data and the Medical insurance claim data (prior to submission to Medical insurance company):
a) The date of service on the claim
b) The Diagnosis code from the Authorization is not present on the claim
c) The CPT code from the Authorization is not present on the claim
d) The claim is being sent to a medical insurance company different from the one that Authorized the procedure.
e) The Authorization code on the claim is different from the Authorization code obtained from the medical insurance.
